(19) United States
(12) Patent Application Publication
(10) Pub. No.: US 2025/0144315 A1
(43) Pub. Date: May 8, 2025

(54) A TUBULAR NEEDLE COVER OF A PEN NEEDLE ASSEMBLY

(71) Applicant: SHL Medical AG, Zug (CH)

(72) Inventor: Sebastian Karlsson, Zug (CH)

(21) Appl. No.: 18/836,920

(22) PCT Filed: Jan. 26, 2023
(86) PCT No.: PCT/EP2023/051857

§ 371 Date: Aug. 8, 2024

(30) Foreign Application Priority Data
Feb. 9, 2022 (EP) 22155846.3

(51) Int. Cl. A61M 5/32; A61M 5/20; A61M 5/34

(52) U.S. Cl. CPC A61M 5/3202; A61M 5/20; A61M 5/346

(57) ABSTRACT

The present disclosure provides a tubular needle cover of a pen needle assembly, the tubular needle cover comprising: a distal sleeve extending along a longitudinal axis between a proximal end and a distal end, wherein the distal sleeve comprises a first fastener; a proximal cover comprising a proximal wall extending in a direction transverse to the longitudinal axis, wherein the proximal cover comprises a second fastener, and wherein the first fastener is attached to the second fastener; and a proximal opening arranged in the proximal wall of the proximal cover, wherein the proximal opening and the distal sleeve are coaxial; and wherein the distal sleeve and the proximal cover are two separate components.

### CROSS-REFERENCE TO RELATED APPLICATIONS

The present application is a U.S. National Phase Application pursuant to 35 U.S.C. § 371 of International Application No. PCT/EP2023/051857 filed Jan. 26, 2023, which claims priority to European Patent Application No. 22155846.3 filed Feb. 9, 2022. The entire disclosure contents of these applications are herewith incorporated by reference into the present application.

### TECHNICAL FIELD

The present disclosure generally relates to a tubular needle cover of a pen needle assembly, and particularly to a tubular needle cover with a proximal cover and a distal sleeve.

### BACKGROUND

Injection devices such as pen type manual injectors or auto-injectors are generally known for the self-administration of a medicament by patients without formal medical training. For example, patients suffering from diabetes may require repeated injections of insulin, or patients may require regular injections of other types of medicaments, such as a growth hormone.

It can be an advantage that medicament containers of the injection devices are spaced apart from the injection needles before an injection operation so that the medicament containers are completely sealed, and the contained medicament can be stored for a longer period. Therefore, it is common that the injection devices are delivered to a user without the injection needles attached; instead, the user will have pen needle assemblies that are configured to be attached to the injection devices, and establish fluid communication with the contained medicament when the user plans to carry out an injection operation. An example of a prior art pen needle assembly is provided in WO2010147552, which provides a device that holds an injection needle with a needle cover that is configured to cover the injection needle before and after injection. Therefore, the user can attach the needle to the injection device safely.

Even though the pen needle assembly as disclosed in the prior art has its advantages, there is still room for improvements. For example, to provide a pen needle assembly that can be manufactured with lower cost

### SUMMARY

The invention is defined by the appended claims, to which reference should now be made.

In the present disclosure, when the term “distal direction” is used, this refers to the direction pointing away from the dose delivery site during use of the injection device. When the term “distal part/end” is used, this refers to the part/end of the delivery device, or the parts/ends of the members thereof, which under use of the injection device is/are located furthest away from the dose delivery site. Correspondingly, when the term “proximal direction” is used, this refers to the direction pointing towards the dose delivery site during use of the injection device. When the term “proximal part/end” is used, this refers to the part/end of the delivery device, or the parts/ends of the members thereof, which under use of the injection device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a tubular needle cover of a pen needle assembly, the tubular needle cover comprising: a distal sleeve extending along a longitudinal axis between a proximal end and a distal end; the distal sleeve comprises a first fastener; and a proximal cover comprising a proximal wall extending in a direction transverse to the longitudinal axis. The proximal cover comprises a second fastener. The first fastener is attached to the second fastener. A proximal opening arranged in the proximal wall of the proximal cover. The proximal opening and the distal sleeve are coaxial. The distal sleeve and the proximal cover are two separate components.

Pen needle assembly commonly refers to an independent needle assembly that is configured to be attached to an injector without needle. As a medicament container of the injector without needle can be fully sealed, this type of injector can be stored for a long period, and also is easier to transport. When a user plans to carry out an injection, the user will attach the pen needle assembly to the injector. The pen needle assembly comprises a needle that will penetrate the medicament container of the injector when the pen needle assembly is attached. A needle cover for preventing the user from accidentally contacting the needle is usually important from the safety aspect. However, if the needle cover is arranged as a part of the injection device, the needle cover may block the pen needle assembly from attaching to the injector, as the needle cover is configured to cover a path of the needle. Thus, a pen needle assembly comprising the needle cover can provide the benefit of safety and ease of use.

Having the distal sleeve and the proximal cover as two separate components provides the benefit of providing a simple manufacturing process. For example, the inner surface of the distal sleeve usually will be arranged with a more complicated structure to perform further functions, such as a needle cover lockout mechanism. To form those structures during the injection molding process, and also to assemble a needle cover to a pen needle assembly without damaging those structures, may request more tools. Thus, the manufacture cost will be increased.

Furthermore, having the distal sleeve and the proximal cover as two separate components can also provide the benefit that the tubular needle cover can be customized with minimized manufacture cost. For example, the distal sleeve and the parts of a pen needle assembly except the tubular needle cover can be standardized, so that the structure of the majority of the parts of the pen needle assembly can be one sophisticated design. The proximal cover can be customized to meet different demands. For example, the length of the proximal cover can be customized dependent on the needle length, injection depth, and/or whether the target injectors have auto-penetration and/or auto-retraction functions or not. The proximal wall of the proximal cover can also be customized with different sensation features, e.g., multiple spikes, or recesses, and/or different contact features, e.g., a flare design, or having a convex surface protruding in the proximal direction, and/or having electronics components, e.g., a skin contact sensor. In a preferred example, the distal sleeve is configured to be attached to the proximal cover during the assembly procedure. Alternatively, the proximal cover can also be attached to the distal sleeve before an injection operation. For example, the proximal cover can be attached to the distal sleeve by a pharmacist and/or a nurse, so that they can, based on the target injectors, attach different proximal covers to the distal sleeve to form the tubular needle cover.

Preferably, according to another embodiment, the proximal opening is the only opening in the proximal wall.

Preferably, according to another embodiment, the proximal wall is convex when looked at from the proximal end of the tubular needle cover.

Preferably, according to another embodiment, the first fastener and the second fastener form a screw thread engagement, a bayonet engagement, or a snap-fit engagement.

Preferably, according to another embodiment, the tubular needle cover is used in a pen needle device.

Preferably, according to another embodiment, the pen needle assembly comprises: a tubular base extending along the longitudinal axis between a proximal end and a distal end. The tubular base comprises a third fastener for attaching to an injector without needle. A needle extending along the longitudinal axis between a proximal end and a distal end, and the needle is attached to the tubular base.

The tubular needle cover is operably connected to the tubular base. The tubular needle cover is axially movable relative to the tubular base between a proximal position where the proximal end of the needle is fully enclosed by the tubular needle cover and a distal position where the proximal end of the needle extends through the proximal opening.

Preferably, according to another embodiment, the tubular base comprises an engaging member extending in a direction transverse to the longitudinal axis from a wall of the tubular base. The tubular needle cover comprises a counter engaging member axially movably connecting to the engaging member of the tubular base, so that the tubular needle cover is axially movable relative to the tubular base between the proximal position and the distal position.

Preferably, according to another embodiment, the third fastener is configured to form a snap-fit engagement with the injection device.

Preferably, according to another embodiment, the third fastener is a snap-fit arm inwardly extending towards the longitudinal axis from the distal end of the tubular base towards proximal end of the tubular base.

Preferably, according to another embodiment, the tubular base comprises a protrusion extending from the distal end of the tubular base. The protrusion is configured to engage with a counter protrusion of the injector to which the third fastener is attached.

Preferably, according to another embodiment, the tubular base comprises a rack extending along the distal end of the base in a circumferential direction. The protrusion is a part of the rack.

Preferably, according to another embodiment, the protrusion extends in the distal direction of the tubular base.

Preferably, according to another embodiment, the pen needle assembly is used in an injection device comprising a multiple-chamber medicament container arranged within the carrier.

Preferably, according to another embodiment, the injection device is an auto-injector or a manual insulin pen.

Preferably, according to another embodiment, the medicament container is a cartridge.

The injection devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the injection devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the injection devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the injection devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a perspective view of a pen needle assembly comprising a tubular needle cover of the invention.

FIG. 2 schematically shows the tubular needle cover of FIG. 1.

FIGS. 3A-B schematically show cross-section views of the pen needle assembly of FIG. 1.

FIG. 4 schematically shows a part of an injection device attached with the pen needle assembly of the invention.

FIGS. 5A-B schematically show perspective views of the injection device of FIG. 4.

FIG. 6 schematically shows a perspective view of the tubular needle cover of FIG. 2 and a tubular base of the pen needle assembly of FIG. 1.

### DETAILED DESCRIPTION

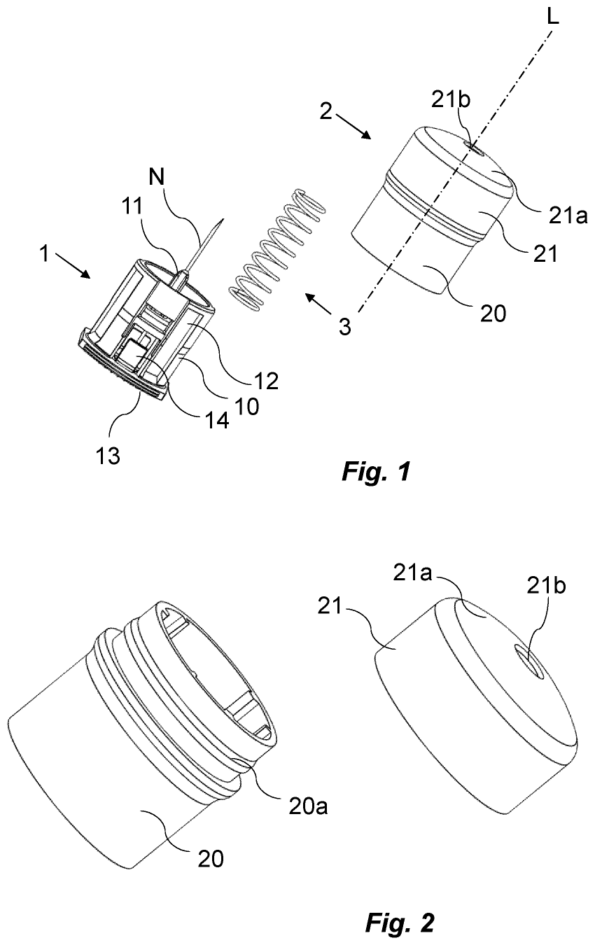

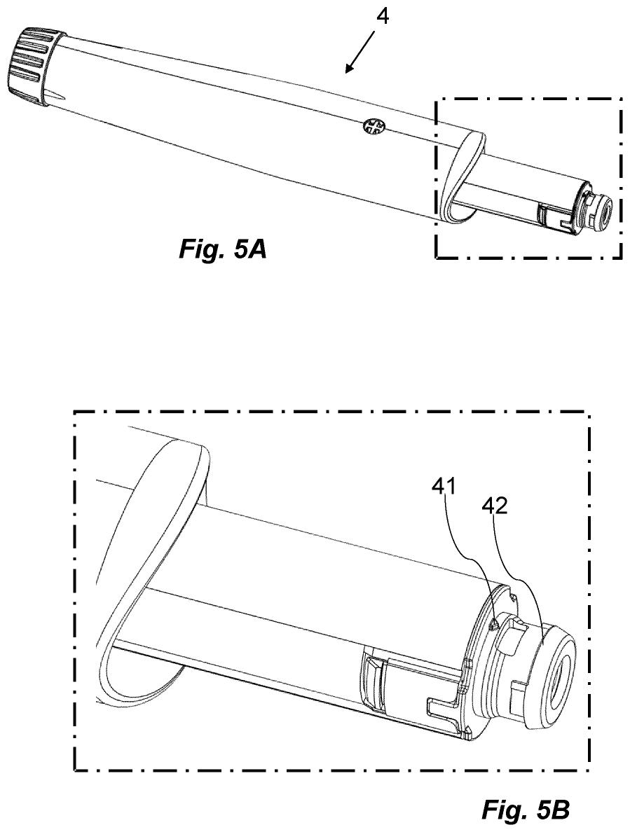

FIGS. 1-6 illustrate a tubular needle cover 2 of a pen needle assembly. The tubular needle cover 2 comprises a distal sleeve 20 extending along a longitudinal axis L between a proximal end and a distal end; and a proximal cover 21 comprising a proximal wall 21a extending in a direction transverse to the longitudinal axis L. The distal sleeve 20 comprises a first fastener 20a and the proximal cover 21 comprises a second fastener 21e. The first fastener 20a is attached to the second fastener 21e, thereby the proximal cover 21 is axially immovable relative to the distal sleeve 20. A proximal opening 21b is arranged in the proximal wall 21a of the proximal cover 21. The proximal opening 21b and the distal sleeve 20 are coaxial. The distal sleeve 20 and the proximal cover 21 are two separate components; preferably, the distal sleeve 20 and the proximal cover 21 are manufactured independently, e.g., manufactured via two separate processes or manufactured at the same time in a separate part of the same mold or with two molds.

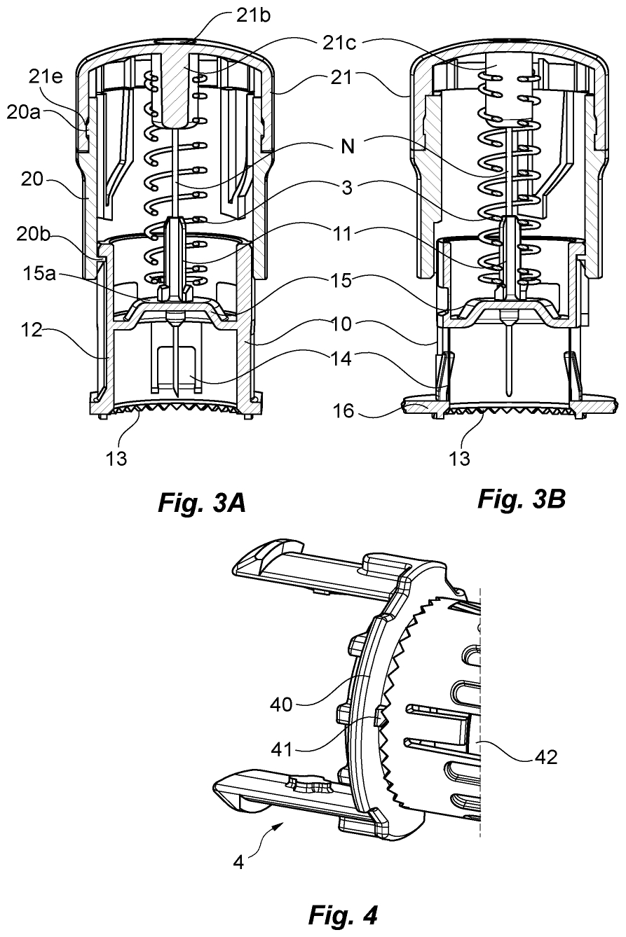

In a preferred example, the distal sleeve 20 is cylindrical. The first fastener can be arranged on an inner surface of the distal sleeve; alternatively, the first fastener 20a can be arranged on an outer surface of the distal sleeve 20 as shown in FIG. 2. Alternatively, the sleeve can be in any suitable shape dependent on the design of the pen needle assembly and/or a targeted injector. In a preferred example, the proximal cover 21 is round for the cylindrical distal sleeve 20. Alternatively, the proximal cover can be any suitable shape dependent on the distal sleeve 20 and/or the pen needle assembly, and/or the targeted injector. In a preferred example, the proximal cover 21 comprises a longitudinal wall extending from the proximal wall 21a towards distal sleeve 20. In a preferred example, the longitudinal wall is an annular wall. In a preferred example, the second fastener is arranged on the inner surface of the longitudinal wall, as shown in FIG. 3A. Alternatively, the second fastener can be arranged on an outer surface of the longitudinal wall or the proximal wall and/or the wall can be two distally extending arms.

In a preferred example, the first fastener 20a and the second fastener 21e can form a screw thread engagement, a bayonet engagement, or a snap-fit engagement. For example, the proximal cover 21 is attached to the distal sleeve 20 via a snap-fit engagement. In one example, the second fastener 21e is a recess 21e configured to be snap-fitted to a protrusion 20a (the first fastener 20a) extending in the direction transverse to the longitudinal axis L, as shown in FIGS. 2-3A. Preferably, the recess 21e is an annular recess and the protrusion 20a is an annular protrusion.

The tubular needle cover 2 is configured to cover a needle N of an injector before an injection operation. Preferably, the tubular needle cover 2 is also configured to cover the needle N after the injection operation. The tubular needle cover 2 can prevent the user from accidentally contacting the needle N, can hide the needle N from a user who is afraid of the needle, and can also make sure the needle is held perpendicular to an injection site so that the correct injection depth can be ensured.

In a preferred example, the proximal opening is the only opening 21b in the proximal wall 21a. As a result, the user will not be confused by seeing multiple internal parts of the pen needle assembly. Also, contamination from users can be prevented from entering into the pen needle assembly.

In a preferred example, the proximal opening 21b is round. In another preferred example, the proximal opening 21b is sized to snugly fit the needle N, meaning that the proximal opening 21b has a diameter slightly greater than a diameter of the needle N. For example, if the outer diameter of the needle is about 0.36 mm (28 gauge needle), the diameter of the proximal opening 21b can be 0.5 mm. Therefore, the user may not accidentally contact the needle if the user presses the proximal wall 21a. This feature will be more important after an injection operation is completed.

In another preferred example, the proximal wall 21a is convex when looked at from the proximal end of the tubular needle cover 2. Thus, the proximal wall 21a can extend the skin of the injection site.

The invention further provides a pen needle assembly that comprises the tubular needle cover 2. The pen needle assembly comprises a tubular base 1 and the tubular needle cover 2. As shown in FIG. 1, the tubular base 1 extends along the longitudinal axis L between a proximal end and a distal end.

The pen needle assembly comprises the needle N extending along the longitudinal axis L between a proximal end and a distal end. The needle is attached to the tubular base 1. The tubular needle cover 2 is operably connected to the tubular base. The tubular needle cover 2 is axially movable relative to the tubular base 1 between a proximal position where the proximal end of the needle N is fully enclosed by the tubular needle cover 2 and a distal position where the proximal end of the needle N extends through the proximal opening 21b.

The tubular base 1 comprises a third fastener 14 for attaching to an injector without needle. As shown in FIG. 4, the third fastener 14 is configured to engage with a counter fastener 42 of the injector 4, thereby attaching the pen needle assembly to the injector 4. In a preferred example, the fastener 14 is configured to form a snap-fit engagement with the counter fastener 42. Thus, a user can simply attach the pen needle assembly onto an injector by pushing the pen needle assembly onto the proximal end of the injector. In another preferred example, the snap-fit arm 14 inwardly extends towards the longitudinal axis L from the distal end of the tubular base 1 towards the proximal end of the tubular base 1.

The tubular base 1 optionally comprises a rack 13 extending along the distal end of the base 1 in the circumferential direction, preferably, the rack 13 extends around the longitudinal axis L. In a preferred example, the rack 13 extends in the distal direction from the distal end of the tubular base 1. The rack 13 is configured to engage with a counter protrusion 41 of the injector 4. Therefore, if the counter fastener 42 of the injector 4 is an annular ledge 42, the engagement between the rack 13 and the counter protrusion 41 can prevent the rotation of the pen needle assembly around the longitudinal axis L when the pen needle assembly is snap-fitted to the injector 4.

It should be noted that, alternatively, the rack 13 can extend in the direction transverse to the longitudinal axis L dependent on the design of the injector. Furthermore, the rack 13 can also be one or more protrusions extending from the distal end of the tubular base 1 dependent on the design of the injector. For example, replacing the rack with one protrusion can provide a guide for the user, so that the user can easily move the pen needle assembly in a predetermined direction to attach the pen needle assembly onto the injector.

In another example, the tubular base 1 comprises a flange 16 extending in the direction transverse to the longitudinal axis L. In a preferred example, the flange 16 extends from the distal end of the tubular base 1. In a preferred example, the rack 13, the fastener 14, and the flange 16 are all arranged to extend from the outer circumferential wall 10. The flange 16 can act as a handle for the user to grip the pen needle assembly so that the user does not need to hold on to the tubular needle cover 2 and might accidentally move the tubular needle cover 2 relative to the tubular base 1.

In a preferred example, the tubular base 1 comprises an inner circumferential wall 11 extending along the longitudinal axis L and around the longitudinal axis L, and an outer circumferential wall 10 extending along the longitudinal axis L and around the longitudinal axis L. The outer circumferential wall 10 surrounds the inner circumferential wall 11. In this example, the inner circumferential wall 11 is configured to surround the needle N and be attached to the needle N. For example, the needle can be glued or welded on an inner surface of the inner circumferential wall 11. The needle N is configured to penetrate an injection site, and is also configured to penetrate a seal of a medicament container when the pen needle assembly is attached to an injector. Specifically, a proximal end of the needle N is configured to penetrate an injection site, and a distal end of the needle N is configured to penetrate the seal of the medicament container.

In one example, the needle N is configured to protrude from both the proximal and distal ends of the inner circumferential wall 11. The needle N is configured to protrude from a proximal end of the outer circumferential wall 10 of the tubular base 1. In a preferred example, the distal end of the outer circumferential wall 10 of the tubular base 1 is configured to surround the distal end of the needle N. In another example, the tubular base 1 comprises a ledge 15 extending in a direction transverse to the longitudinal axis L between the outer circumferential wall 10 and the inner circumferential wall 11.

It should be noted that, instead of the circumferential walls, the tubular base can be formed in any suitable shape depending on the design of the injector. For example, the outer wall can be formed by two elongated arms extending parallel to the inner wall. Alternatively, instead of the outer circumferential wall, the tubular base may comprise only the inner circumferential wall.

When the tubular needle cover 2 is attached to the tubular base 1, the proximal opening 21b is configured to be coaxial relative to the tubular base 1, so that the needle N can pass through the proximal opening 21b for an injection operation.

The tubular base 1 comprises an engaging member 12 extending in the direction transverse to the longitudinal axis L from the wall of the tubular base 1. The engaging member 12 is configured to movably engage the tubular needle cover 2. In a preferred example, the engaging member 12 is arranged in the outer circumferential wall 10 of the tubular base 1.

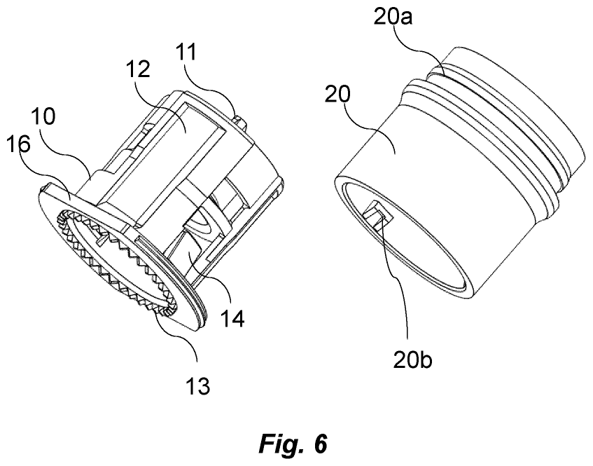

The distal sleeve 20 comprises a counter engaging member 20b axially movably connected to the engaging member 12 of the tubular base 1. In a preferred example, the engaging member 12 is a groove, and the counter engaging member 20b is a protrusion 20b, as shown in FIG. 6. The groove 12 of the tubular base 1 opens in the direction transverse to the longitudinal axis L, and the protrusion 20b of the needle cover 2 extends in the direction transverse to the longitudinal axis L and protrudes into the groove 12. Therefore, the tubular needle cover 2 is axially movable relative to the tubular base 1 between a proximal position and a distal position. When the tubular needle cover 2 is in the proximal position, the tubular needle cover 2 is fully enclose a proximal end of the needle N. When the tubular needle cover 2 is in the distal position, the proximal end of the needle N passes through the proximal opening 21a. In a preferred example, the exact proximal position and the distal position can be defined by a proximal end and a distal end of the groove 12, meaning that the proximal end of the groove 12 blocks any further proximal movement of the protrusion 20b and the distal end of the groove 12 blocks any further distal movement of the protrusion 20b, thereby blocking the axial movement of the tubular needle cover 2 beyond the predetermined distal and proximal position. The exact proximal and distal positions are dependent on the design of the pen needle assembly, e.g., the length of the needle. It should be noted that the engaging member can also be a slot. Alternatively, the engaging member can be a protrusion and the counter engaging member can be a groove or a slot.

In a preferred example, the tubular base 1 is at least partially arranged within the tubular needle cover 2. In this example, the engaging member 12 of the tubular base 2 is arranged on an outer wall of the tubular base, in a preferred example, on the outer circumferential wall 10. The counter engaging member 20b of the tubular needle cover 2 is arranged on an inner wall of the distal sleeve 20.

In a preferred example, the pen needle assembly comprises a biasing member 3 extending in the direction of the longitudinal axis L between a proximally directed surface 15a of the tubular base 1 and a distally directed surface 21d of the tubular needle cover 2. In a preferred example, the distally directed surface 21d of the tubular needle cover 2 is defined by the proximal wall 21a of the tubular needle cover 2. In another example, the proximally directed surface 15a of the tubular base 1 is defined by the ledge 15. In a preferred example, the biasing member 3 is a coil spring. In this example, the proximal cover 21 comprises a tube 21c extending from the proximal wall 21a. The tube 21c is configured to be coaxial relative to the needle N. The coil spring 3 surrounds the tube 21c of the proximal cover 21 and surrounds the inner circumferential wall 11 so that the tube 21c and the inner circumferential wall 11 can prevent the coil spring from buckling. Alternatively, the proximally directed surface can be defined by a protrusion extending from the inner circumferential body in the direction transverse to the longitudinal axis L. Alternatively, the coil spring can be replaced by a flexible arm.

The biasing member 3 is configured to bias the tubular needle cover 2 to move between the proximal position and the distal position so that the tubular needle cover 2 will surround the needle N before and after the injection operation. It is preferable that the pen needle assembly comprises a needle cover lockout mechanism for locking the tubular needle cover in the proximal position after the injection operation. There are many different variations of needle cover lockout mechanism that can be realized together with the pen needle assembly of the invention, e.g., WO2010147552 or WO2010126432 (both of which are hereby incorporated by reference), so this feature will not be discussed in any further detail herein. The needle cover lockout mechanism can be arranged between the tubular needle cover 2 and the tubular base 1. Preferably, the needle cover lockout mechanism is arranged between the distal sleeve 20 of the tubular needle cover 2 and the outer circumferential wall 10 of the tubular base 1. It should be noted that the needle cover lockout mechanism can be desirable if the user does not have any professional training and/or the injection operation is not carried out in a professional medical setting, e.g., a hospital, so that any injury from a used needle can be prevented.

Furthermore, the pen needle assembly optionally comprises a packaging. In one example, the tubular base 1 and the tubular needle cover 2 are contained within the packaging. In this example, the packaging can be a rigid shell with a seal membrane. Alternatively, the packaging can be a removable strip wrapped between the tubular needle cover and the tubular base. The removable strip is configured to block an axial movement between the tubular needle cover and the tubular base. In this example, the packaging comprises a cover for removably sealing the proximal opening of the tubular needle cover. There are many different variations of packages that can be realized together with the pen needle assembly of the invention, e.g., WO2016055241 (which is hereby incorporated by reference), so this feature will not be discussed in any further detail herein.

The inventive concept has mainly been described above with reference to a few examples. However, as is readily appreciated by a person skilled in the art, other embodiments than the ones disclosed above are equally possible within the scope of the inventive concept, as defined by the appended claims.

1-14. (canceled)

15. A tubular needle cover comprising:
a distal sleeve extending along a longitudinal axis between a proximal end and a distal end, wherein the distal sleeve comprises a first fastener;
a proximal cover comprising a proximal wall extending in a direction transverse to the longitudinal axis, wherein the proximal cover comprises a second fastener, and wherein the first fastener is attached to the second fastener; and
a proximal opening arranged in the proximal wall of the proximal cover, wherein the proximal opening and the distal sleeve are coaxial, and wherein the distal sleeve and the proximal cover are two separate components.

16. The tubular needle cover according to claim 15, wherein the proximal opening is the only opening in the proximal wall.

17. The tubular needle cover according to claim 15, wherein the proximal wall is convex when looked at from the proximal end of the tubular needle cover.

18. The tubular needle cover according to claim 15, wherein the first fastener and the second fastener form a screw thread engagement, a bayonet engagement, or a snap-fit engagement.

19. A pen needle assembly comprising the tubular needle cover according to claim 15.

20. The pen needle assembly according to claim 19, wherein the pen needle assembly comprises:
a tubular base extending along the longitudinal axis between a proximal end and a distal end; wherein the tubular base comprises a third fastener for attaching to an injector without needle; and
a needle extending along the longitudinal axis between a proximal end and a distal end; wherein the needle is attached to the tubular base,
wherein the tubular needle cover is operably connected to the tubular base; and wherein the tubular needle cover is axially movable relative to the tubular base between a proximal position where the proximal end of the needle is fully enclosed by the tubular needle cover and a distal position where the proximal end of the needle extends through the proximal opening.

21. The pen needle assembly according to claim 20, wherein the tubular base comprises an engaging member extending in a direction transverse to the longitudinal axis from a wall of the tubular base, wherein the tubular needle cover comprises a counter engaging member axially movably connecting to the engaging member of the tubular base, so that the tubular needle cover is axially movable relative to the tubular base between the proximal position and the distal position.

22. The pen needle assembly according to claim 20, wherein the third fastener is configured to form a snap-fit engagement with the injection device.

23. The pen needle assembly according to claim 20, wherein the third fastener is a snap-fit arm inwardly extending towards the longitudinal axis from the distal end of the tubular base towards proximal end of the tubular base.

24. The pen needle assembly according to claim 23, wherein the tubular base comprises a protrusion extending from the distal end of the tubular base, and wherein the protrusion is configured to engage with a counter protrusion of the injector to which the third fastener is attached.

25. The pen needle assembly according to claim 24, wherein the tubular base comprises a rack extending along the distal end of the base in a circumferential direction; and wherein the protrusion is a part of the rack.

26. The pen needle assembly according to claim 24, wherein the protrusion extends in the distal direction of the tubular base.

27. An injector comprising the pen needle assembly of claim 20.

28. The injector of claim 27, wherein the injector is an autoinjector or a manual insulin pen.
